(19) United States
(12) Patent Application Publication
(10) Pub. No.: US 2016/0310467 A1
(43) Pub. Date: Oct. 27, 2016

(54) ONDANSETRONE TO REVERSAL OF THE SOFT TISSUE ANESTHESIA

(71) Applicant: Apiliogullari SEZA, Konya (TR)

(72) Inventor: Apiliogullari SEZA, Konya (TR)

(21) Appl. No.: 15/150,437

(22) Filed: May 10, 2016

Related U.S. Application Data
(62) Division of application No. 14/438,227, filed on Apr. 23, 2015.

(51) Int. Cl. A61K 31/4178

(52) U.S. Cl. CPC A61K 31/4178

(57) ABSTRACT

Invention is about the ondansetrone hydrochloride which is used in the reversal of the effect of the local anesthesia of the soft tissues in a short time period especially in dental procedures.

### CROSS REFERENCE TO RELATED APPLICATION

This is a division of application Ser. No. 14/438,227 filed on Apr. 23, 2015.

### TECHNICAL FIELD

Invention is about the ondansetrone hydrochloride which is used in the reversal of effect of the local anesthesia on the soft tissues in a short tune period especially in dental procedures.

### PRIOR ART

To provide pain control by injecting the local anesthetic agents is a routine part of the daily dental procedures. The most important disadvantage of that implementation is extensibility of the local anesthesia of the soft tissues (lip and tongue) can reach up to five hours but the dental procedures are mostly completed in one hour or sooner. The extended soft tissue anesthesia can cause of biting the lips, tongue or cheeks by accident especially in kid patients. And in adults, it may cause speech disorder, shape disorders in the face of the patient and skipping meals because of being unable to eat in diabetic patients and the risk of hypoglycemia. Eating hot meals and drinking hot beverages can cause being irritated in the mouth by the time local anesthesia continues for both children and adults. Besides effecting the daily life in negative way, that negative situations can cause serious health issues such as coma, even death for the diabetic patients.

### SHORT DESCRIPTION OF THE INVENTION

The reversal of the effect of the local anesthetic agent in a short time is important in dental procedures. Ondansetrone is a strong antiemetic for preventing and treating nausea and vomiting in perioperative period. It is stated that besides preventing nausea and vomiting, intravenous implementations of ondansetrone can reduce the postoperative analgesic efficiency of the tramadole, the itchiness caused by using spinal or epidural morphine, the analgesic efficiency of the opioids and the sensory block level caused by spinal anesthesia which is established by local anesthetics.

The effect of the five hydroxytriptamine (5-HT3) to the pain activity is different. Both analgesic and hyperalgesic effects are reported. It is stated that the ondansetrone which is a selective antagonize of 5-HT3 has local anesthetic features. Besides, it is claimed in the scientific studies that the ondansetrone accelerates the regression of the sensorial block level in subarachnoid anesthesia by lidocaine, and it has no effect in sensorial block level in subarachnoid anesthesia by ropivacaine. And also it is known that the granisetron can accelerates the reversal of the sensorial block caused by spinal anesthesia with bupivacaine.

### DETAILED DESCRIPTION OF THE INVENTION

Invention is about the reversal of the effect of the local anesthetic applied to the soft tissue, in a short time period by applying ondansetrone, which is a 5-hydroxytriptamine (5-HT3) antagonist, to the same area. And it is also about the removal of the problems encountered by local anesthesia.

Anesthesia is a procedure for the patient before the surgery which prevents the feeling of the pain during the operation. Only the surgery area is anesthetized with the local anesthesia. Various kind of local anesthetics are used for local anesthesia. In the recent years, depending on the technological development of medicines and equipments for anesthesia, complications are significantly reduced. Since 1860, the usage of local anesthetics has provided easiness, safety and economical advantage to the surgery.

Anesthesia implementations can be classified as local anesthesia, regional anesthesia and general anesthesia. Local anesthesia is the temporarily blockage of carrying the painful stimulations by nerve tracks. Only the surgery area is anesthetized with the local anesthesia. Patient is awake, conscious, hears the voices, can feel the press, pressure or sonic movements but does not feel any pain. Local anesthesia effect can last from as few hours up to five hours. During that period, the problems that patient has to face because of being effected by local anesthesia are described in detail in the prior art section. The solution of these problems is reversal of the effect of the local anesthesia by the doctor in a short time after finishing the operation. Because of the effects of the local anesthetics can take hours to be removed by their selves. In the studies of mine about reversal of that local anesthesia effect, ondansetrone, which is a 5-hidroksitriptamin (5-HT3) antagonist, is implemented to the same area according to patient's situation (age, weight, sensitivity etc.) and to type and dosage of local anesthetic were local anesthesia is applied. It antagonizes the effect of the local anesthetic in a short time (a few minutes) and provides the patient to remain his/her daily life. In addition, there are patients who are obligated to stay in the hospital until they are fully recovered from the effects of the local anesthesia. With our implementation, the discharging from hospital of these patients in a very short time, will be provided. This situation will remove the costs and difficulties for the hospital and for the patient. Especially in dental procedures, the effect of the local anesthetics which lasts for hours can be removed in minutes with this implementation and patient is provided to remain his/her daily life in comfort.

1. A method of anesthetizing a local area, the method comprising the steps of:
applying an anesthetic to the local area; and
applying an agent for reversal of anesthesia to the local area;
wherein the agent comprises ondansetrone or ondansetrone hydrochloride.

2. The method of claim 1, wherein the agent is applied to the local area after the anesthetic is applied to the local area.

3. The method of claim 1, wherein the agent is applied to the local area according to a patient's situation including at least one of age, weight and sensitivity.

4. The method of claim 1, wherein the agent is applied to the local area according to the type and dosage of the anesthetic.
